(19) United States
(12) Patent Application Publication
(10) Pub. No.: US 2010/0041160 A1
(43) Pub. Date: Feb. 18, 2010

(54) ANALYSIS OF HIGH MOLECULAR WEIGHT MPEG ALCOHOL COMPOSITIONS

(76) Inventors: Chris Chappelow, Midland, MI (US); Edward D. Daugs, Midland, MI (US); Sterling C. Gatling, Midland, MI (US); Kevin D. Sikkema, Midland, MI (US); Maciej Turowski, Midland, MI (US); Ross A. Wallingford, Scott Depot, WV (US)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/603,708

(22) Filed: Oct. 22, 2009

Related U.S. Application Data
(62) Division of application No. 10/932,629, filed on Sep. 1, 2004.

(51) Int. Cl. G01N 33/44

(52) U.S. Cl. 436/85

(57) ABSTRACT

The invention is directed toward novel high molecular weight and high purity mPEG alcohol compositions as well as a process for obtaining said compositions by removing PEG diols from the mPEG alcohol after polymerization is complete.

### BACKGROUND OF THE INVENTION

1. Field of the Invention

The invention is directed toward novel high molecular weight and high purity mPEG alcohol compositions as well as a process for obtaining said compositions by removing PEG diols from the M-PEG alcohol.

2. Description of the Prior Art

The therapeutic efficacy of bioactive molecules can be improved by conjugating them with poly(ethylene glycol)(PEG). The PEG is often a linear poly(ethylene glycol) with one hydroxyl end group capped with a methyl group and the other hydroxyl group activated for conjugation. An activated mPEG is made from mPEG alcohol, which in turn is typically made by initiating anionic polymerization of ethylene oxide with methanol or its equivalent. If there is any water in the polymerization, it forms a linear PEG with hydroxyl groups on both ends. Since the PEG diol undergoes the same activation and conjugation chemistry as mPEG alcohol, it's presence in the mPEG alcohol is undesirable.

The amount of PEG diol can be reduced by decreasing the amount of water in the polymerization reactor. U.S. Pat. No. 6,455,639, discloses the production of M-PEG alcohol by polymerization of EO under very dry conditions with molecular weights up to 20,861. Obtaining these very low levels of water requires great effort.

Alternatively, the PEG diol can be converted to its unreactive dimethyl ether. This is performed by initiating polymerization of EO with benzyl alcohol, permethylating all the hydroxyl groups (both on the benzyl PEG and PEG diol), and then removing the benzyl group to give mPEG alcohol and dimethyl PEG (U.S. Pat. No. 6,448,369). The permethylation of the PEG diol requires two additional chemistry steps, and the concentration of the desired mPEG alcohol is reduced by the presence of the dimethyl PEG.

In addition to the processes described above, a variety of purification techniques for removal of excess diol have been described in the literature. Snow (Snow U.S. Pat. No. 5,298,410, 1994) converted all the hydroxyl groups to dimethoxytrityl ethers, separated the ditrityl PEG from the methyl trityl PEG by reverse phase chromatography, and then removed the trityl group from the methyl trityl PEG to give mPEG. Lapienis (Lapienis and Penczek, J. Bioactive Compatible Polymers, 16, 206 (2001)) used ultrafiltration to purify 2K mPEG, although analysis indicated that little if any PEG was removed. Kazanskii (Kazanskii et al, Polymer Science Ser. A, 42, 585 (2000)) also used ultrafiltration to remove impurities. Kokufuta (Kokufuta et al, Polymer, 24, 1031 (1983)) describes the narrowing of the molecular weight distribution of PEG by complexing it with polyacrylic acid (PAA).

All prior art purifications cited above use mPEG alcohol with a molecular weight of 5 kDaltons or less. The longevity of bioactive molecules attached to PEG increases with the molecular weight of the PEG. Therefore, it is desirable to use activated mPEG alcohols with molecular weights of at least 10 kdaltons.

### SUMMARY OF THE INVENTION

The invention is directed toward novel high molecular weight and high purity mPEG alcohol compositions as well as a process for obtaining said compositions by using separation techniques to remove PEG diols from the mPEG.

### DETAILED DESCRIPTION OF THE PREFERRED EMBODIMENTS

The invention comprises a monomethoxy poly(ethyleneglycol) of at least 95% chemical purity by weight, having a polydispersity value of less than 1.1 and having a defined molecular weight of from 10,000 Daltons to about 60,000 Daltons. Preferably, the monomethoxy poly(ethyleneglycol) of the invention has a polydispersity value of less than 1.05. The invention further comprises a process for obtaining a monomethoxy poly(ethyleneglycol) of at least 95% chemical purity by weight, having a polydispersity value of less than 1.1 and having a defined molecular weight of at least 10,000 Daltons and up to around 60,000 Daltons. The process comprises a first step of providing an impure monomethoxy poly(ethyleneglycol) characterized as a monomethoxy poly(ethyleneglycol) having one or more impurities including poly(ethyleneglycol) [hereinafter “PEG diol”] and low molecular weight organic and inorganic molecules. The impure monomethoxy poly(ethyleneglycol) can be obtained according to well-known polymerization techniques as described in “Poly(Ethylene Oxide)” (F. E. Bailey, Jr. and J. V. Koleske, Academic Press, New York, 1976).

The impure monomethoxy poly(ethyleneglycol) is directly purified by means of one or more separation techniques such as, but not limited to, polymeric adsorption/desorption, ultrafiltration, chromatography, precipitation or combinations of one or more of the above. The separated PEG diol and low molecular weight organic or inorganic molecules are then removed from the purified monomethoxy poly(ethyleneglycol). The PEG diol may be either of higher or of lower molecular weight than the purified monomethoxy poly(ethyleneglycol) thereby obtained.

In one embodiment of the invention, the separation technique comprises polymeric adsorption/desorption. The polymeric adsorption/desorption preferably comprises treatment of the impure mPEG alcohol with a polymer containing repeating pendant functional groups capable of hydrogen bonding with the ether oxygen atoms of mPEG alcohol and/or PEG diol, in the presence of a protic solvent. Preferably, the pendant functional groups are selected from the group consisting of CO₂H, SO₃H, PO₃H₂, NH, NH₂, OH and SH. Preferably, the polymer is a polyacid. More preferably, the polymer is a poly(carboxylic acid). Most preferably, the polymer is a crosslinked poly(carboxylic acid) resin. Preferably, the protic solvent is selected from the group comprising water, a C₁₋₃ alcohol or a mixture thereof. More preferably, the protic solvent is water.

In a second embodiment of the invention. The separation technique comprises ultrafiltration. Ultrafiltration comprises contacting an impure mPEG alcohol solution with a membrane of the appropriate pore size as to allow materials of lower molecular weight to pass through the membrane and be removed.

The separation technique of chromatography comprises placing the polymer on one end of a column packed with an active support, passing a suitable solvent through the column, and collecting fractions at the other end of the column. The various components of the impure alcohol are separated on the column and collected in separate fractions.

Analysis of the mPEG polymer for PEG diol content is determined by critical condition HPLC analysis (Gorshkov; J. Chrom. 523, 91 (1990); Kazanskii et al, Polymer Science Ser. A, 42, 585 (2000); Lapienis and Penczek, J. Bioactive Biocompat Polymers, 16, 206 (2001). Critical condition chromatography is useful in this application for analytical separation of the mPEG from PEG diol as the retention time of the polymer is independent of molecular weight, and is only a function of polymer end groups. Specifically in this case, the mPEG and PEG diol polymers are derivatized with 3,5-dinitrobenzoyl chloride and separated at the critical point on a reversed phase analytical column with UV detection.

The separation technique of precipitation comprises the successive precipitation of polymer from a solution by addition of a miscible nonsolvent, by controlled cooling, or by controlled evaporation of solvent. The polymer molecules with higher molecular weight precipitate first.

In a preferred embodiment of the invention, the process further includes the step of isolating the pure monomethoxy poly(ethyleneglycol) composition from aqueous solution by an isolation technique selected from the group consisting of spray drying, addition of a non-solvent, extraction into a good solvent followed by addition of a non-solvent and evaporation of solvent under vacuum. The more preferred isolation technique comprises spray drying. The step of spray drying comprises spraying a solution of polymer into a chamber to form droplets, the solvent of which is evaporated in a flow of hot air to give a dry powder.

### Examples

### Example 1

### Ultrafiltration

Removal of Low Molecular Weight Polymer from 30K mPEG

A 3.8 kg sample of crude mPEG (Mp 31,491, 5.0 mol % diol) was dissolved in about 75 kg of DI water and loaded to the ultrafiltration feed tank. An Osmonics 2.5 m² 10K MWCO membrane (model #PW2540F1080) was installed. The recirculation pump was turned on at 28% output. The retentate and permeate back pressure valves were adjusted to achieve a retentate flowrate of 15 lpm with a 30 psi transmembrane pressure. The tank volume was initially concentrated down to about 40 liters, at which time DI water was continuously added in order to maintain a constant tank volume. A total of 303 kg of permeate was collected at an average rate of about 0.4 lpm. GPC analysis of a composite sample indicated the permeate contained 0.7 kg of mPEG. The GPC profile of the permeate was noticeably skewed to the lower molecular weight material. The retentate fraction in the feed tank was further concentrated to about 33 liters and then drained through a 0.2 micron polypropylene polish filter. The final 32.9 kg retentate sample contained 7.6% mPEG by GPC (2.5 kg mPEG). DI water was loaded to the feed tank and recirculated for about 15 minutes to rinse the membrane and piping. GPC analysis indicated the 36.3 kg rinse sample contained an additional 0.6 kg of mPEG. The mPEG in the final retentate sample was isolated using a spray dryer. The diol concentration in the final isolated product was 2.7 mol %.

### Example 2

### Polyacrylic Acid (PAA)

Removal of High Molecular Component at Ambient Temperature from 20K mPEG

Crude 20 kDa mPEG was dissolved in DI water to make a 1.49 wt % solution of mPEG. 317.3 g of this solution were added to a 1-L Erlenmeyer flask fitted with a mechanical stirrer. While stirring, a total of 21 g of Dowex MAC-3 PAA ion exchange resin (containing 48 wt % water) were added. The reaction was stirred at 25° C. for 42 hours. The resin was filtered. GPC analysis of the corresponding filtrate indicated that the high molecular weight component was reduced from 8.6 to 0.3 area %.

Removal of High Molecular Component at Higher Temperature from 20K mPEG

Crude 20 kDa mPEG was dissolved in DI water to make a 1.19 wt % solution of mPEG. 571.2 g of this solution were added to a 1-L round bottom flask fitted with a water recirculation bath, mechanical stirrer, condenser, and N₂ purge. While stirring, 22.4 g of Dowex MAC-3 PAA ion exchange resin (containing ˜50 wt % water) and 0.083 g of hydroquinone were added. The reaction was stirred at 63° C. for 4.3 hours. The resin was filtered. GPC analysis of the corresponding filtrate indicated that the high molecular weight component was reduced from 9.2 to 0.6 area %.

Removal of High and Low Molecular Components from 20K mPEG

8017.1 g of 0.725 % aqueous mPeg were added to a 12 litter round bottom flask equipped with a mechanical stirrer, condenser, temperature controller, and N₂ purge. While stirring, 93 g of Dowex MAC-3 PAA (contain ˜50% water) and 1.4 g of hydroquinone were added. The reaction was stirred at 56° C. for 39 hours. The resin containing the high molecular PEG component was separated by filtration and discarded. 7900 g of filtrate containing 31 g of mPEG were collected and GPC analysis showed the high molecular weight component was reduced from 4.6 to 0.2 area %.

The filtrate was added back to the reactor along with 91 g of fresh PAA (enough to complex greater than 75% of the mPEG). The reaction mixture was stirred at 61° C. for 32 hours. The PAA resin containing the mPEG was collected by filtration and the filtrate (7872 g) was discarded. 115 g of the PAA resin wetcake (containing mPEG) were washed with deionized water and added back to the reactor along with 237 g of 30% aqueous tetrahydrofuran (THF). The mixture was stirred at 25° C. for 20 hours. The PAA resin, from which the mPEG had now been removed, was separated from the mPEG solution by filtration and discarded. GPC analysis of the filtrate showed the low molecular weight component was reduced from 4.0 to 0.7%. THF was removed from the filtrate and 14.8 g mPEG was isolated by extracting the filtrate with chloroform.

### Example 3

### Spray Drying

A Buchi B-191 Mini Spray dryer was set up with the following operating parameters: nitrogen flow was 700 L/h, inlet temperature was 95° C., vacuum aspirator was 50% of the maximum speed, and DI water was fed at 15% of the maximum rate. After the system was equilibrated for 30 minutes, the outlet temperature was 36° C. A 951-g aqueous solution containing 3.0 wt % of mPEG (28.5 g) was loaded at 15% of the maximum rate. Over the course of the 3 hour and 10 minute addition, the inlet temperature was adjusted to 97, then 99° C. The outlet temperature ranged from 36 to 38° C. A total of 9.5 g of mPEG was collected as a fluffy white powder from the cyclone. The mPEG contained 0.31 wt % water by Karl Fisher titration.

### Example 4

### PAA, Ultrafiltration, and Spray Drying

A sample of M-PEG (Mp 28164, 3.6 mol % PEG diol) was treated with PAA as described above to provide 15.2-kg of an aqueous solution containing 92.7 g of polymer. The solution was subjected to ultrafiltration using an Osmonics 10K MWCO polyethersulfone membrane as described above to provide a 3.2-kg aqueous solution containing 67.7 g of polymer. A portion of the aqueous solution was spray dried as described above to provide 9.1 g of mPEG polymer (Mp 29178) containing 1.3 mol % of PEG diol.

1-14. (canceled)

15. A method for analyzing a polyethylene glycol diol content in a monomethoxy poly(ethylene glycol) polymer, comprising reacting a polymer mixture to form a derivative and separating using critical condition HPLC on a reversed phase column.

16. The method of claim 15, wherein a monomethoxy poly(ethylene glycol) polymer has a molecular weight from 10,000 to 60,000 Daltons.

17. The method of claim 15, wherein a polymer mixture is reacted with 3,5-dinitrobenzoyl chloride.
